(19) United States
(12) Patent Application Publication
(10) Pub. No.: US 2017/0049996 A1
(43) Pub. Date: Feb. 23, 2017

(54) ANTI-BEND CATHETER BRACE

(71) Applicant: Susan Klinkner, Manitowoc, WI (US)

(72) Inventor: Susan Klinkner, Manitowoc, WI (US)

(21) Appl. No.: 14/829,247

(22) Filed: Aug. 18, 2015

(51) Int. Cl. A61M 25/02

(52) U.S. Cl. CPC A61M 25/02; A61M 2025/0266; A61M 2025/026

(57) ABSTRACT

An anti-bend catheter brace includes a disk-like pad having a peripheral edge therearound, a topside and a substantially planar underside, and strapping configured to hold the pad over tissue surrounding the subcutaneous catheter while maintaining substantial planarity of the underside and a region of tissue thereunder.

### FIELD OF THE INVENTION

This invention relates to intravenous infusion, and, more specifically, to the field of peripheral catheter placement at the antecubital fossa.

### BACKGROUND OF THE INVENTION

In the field of intravenous infusion, the antecubital fossa is a known useful site for percutaneous placement of a peripheral catheter, providing convenient bedside access to suitable blood vessels in a patient's arm. While alternative useful sites are known and, in some instances, may be preferred to the antecubital fossa, in some circumstances the antecubital fossa is an especially advantageous site, including for reasons of a patient's particular anatomy. Indeed use of the antecubital fossa is common despite known risks of complication associated with peripheral catheter insertion at such site, the mitigation of which is an object of this invention.

Peripheral catheter placement at the antecubital fossa is known to be associated with risks of complication relating to patient arm movement, particularly elbow flexion. Notable complications that can be caused by elbow flexion include, for example, functionally-significant bending, kinking and related occlusion of flow in a subcutaneous catheter, in turn raising further medical risks. Such functionally-significant bending, kinking and occlusion, even if brief, risks distressing the patient and places a burden of intervention upon care-givers, such as busy nurses. Other notable complications related to elbow flexion in patients having peripheral catheter placement at the antecubital fossa include, for example, tissue irritation occasioned by catheter movement against blood vessel walls. Need for an apparatus and method preventing or at least maximally mitigating catheter bending, and thus the associated risks, is apparent.

Some health care professionals attempt to reduce such risks simply by instructing patients to refrain from arm movement, but such instructions do not provide any actual solution to the problem, because patient arm movements including elbow flexion still frequently occur whether unintentionally (e.g., during sleep) or intentionally (e.g., in reaction to the inevitable discomfort of prolonged non-movement, or for reaching purposes).

Other ways by which those in the field have sought to address the subject problem include use of specialized catheters or related accessories such as the “non-kinking” catheter of U.S. Pat. No. 8,641,677 and the “anti-kinking” device of U.S. Pat. No. 7,198,066. Such devices have disadvantages, including that they do not address the full range of known complications associated with elbow flexion in patients receiving intravenous fluids at the antecubital fossa (for example, tissue irritation), and in that they involve use of alternative equipment immediately in the insertion site (as opposed to around it).

The prior art device of U.S. Pat. No. 9,039,663 addresses elbow flexion specifically but by providing a device of structure and function which are highly disadvantageous in comparison to the instant invention—for example by facilitating and guiding subcutaneous catheter bending, rather than inhibiting subcutaneous catheter bending, as in the instant invention, and by eliminating access/observation points around the insertion site rather than providing access/observation points around the insertion site, as in the instant invention. Even short of sharp kinking, bending has been observed to restrict flow in some catheters, so inhibiting bending is to be preferred.

Because the known complications of using a bend-prone insertion site such as the antecubital fossa are caused by movement of tissue immediately surrounding the inserted catheter relative to the inserted catheter, a solution to the problem involves prevention of such relative movement.

The instant invention is based in part on recognition that, given the structure and ample presence of soft tissue in the antecubital fossa of a typical patient, such site is typically pliable and, accordingly, susceptible to unidirectional compression by a pushing force applied from a point external to the patient's arm to a point of contact with an external surface of the site. Upon application of such force, such compression typically occurs notwithstanding the presence of an inserted catheter and related accessories known to those in the field of intravenous infusion (e.g., transparent film dressing overlaying the site and securing a clean field thereupon). When applied to an area of bend-prone tissue in which a catheter is subcutaneously present, a moderate and regular unidirectional pushing force, applied across a substantially planar area, non-injuriously compresses the tissue around the inserted catheter, creates a non-bending plane of tissue around the inserted catheter, and thereby restricts the movement of such tissue relative to the inserted catheter, including when elbow flexion or other bodily movement occurs, without causing obstruction of flow in the catheter.

An object of this invention is to provide an apparatus and method by which such force can be applied.

A related object of this invention is to provide an apparatus and method which, by distributing such force across a substantially planar area, presents no impediment to circulation in the arm.

A further object of this invention is to provide such an apparatus and method, wherein the apparatus is non-adhesively securable/secured to the patient's arm about the site such that the apparatus may easily be fitted around the site and removed therefrom without disturbing other materials present, e.g., without affecting position or securement of adhesively-secured dressing and without jeopardizing clean-field integrity at such site.

Another object of this invention is to provide such an apparatus and method, wherein the apparatus may be adjusted to accommodate various arm sizes and to generate various desired levels of force/compression.

An additional object of this invention is to provide such an apparatus and method that may be used during various tests and procedures, including without limitation Magnetic Resonance Imaging (MRI).

Yet another object of this invention is to provide an apparatus and method that increases comfort, security and general freedom of movement for patients receiving intravenous infusion at bend-prone insertion sites such as the antecubital fossa.

Another object of this invention is to provide an apparatus and method that prevents occlusion-related interruption of automated intravenous drug and fluid infusion systems. A related object of this invention is to reduce the current level of demand for manual intervention by care-givers of patients receiving intravenous infusion, which represents a significant and undesirable burden among health care professionals on hospital floors.

Still another object of this invention is to provide an apparatus and method that leaves adjacent arm surfaces uncovered, thus facilitating access/observation of the insertion site and increasing patient comfort while the apparatus is secured to the patient's arm.

These and other objects of the invention will be apparent from the following descriptions and from the drawings.

### SUMMARY OF THE INVENTION

This invention includes apparatus and method of using such apparatus which overcome shortcomings in the prior art in the field and achieve the objectives alluded to above. Of particular note is that the apparatus and method of this invention bring increased safety, comfort and freedom of movement to medical patients receiving fluids intravenously, and correspondingly ease burdens of intervention for professional care-givers of such patients.

The apparatus of this invention is a brace for inhibiting subcutaneous bending of an inserted catheter. The brace includes a disk-like pad including a peripheral edge therearound, a topside and a substantially planar underside and strapping configured to hold the pad over tissue surrounding the subcutaneous catheter while maintaining substantial planarity of the underside and a region of tissue thereunder.

In certain preferred embodiments of this invention, the brace is adapted for use with a human arm and is movable between an unmounted position in which the brace is not attached to the arm and a mounted position in which it is secured to the arm with the pad at the antecubital fossa of the arm.

For embodiments adapted for use with a human arm, it is preferred that, in the mounted position, the pad has lower-arm-adjacent portion abutting an adjacent region of the lower arm at a lower-arm pad-abutting point not covered by the brace; an upper-arm-adjacent portion abutting an adjacent region of the upper arm at an upper-arm pad-abutting point not covered by the brace; and at least one lateral portion abutting adjacent region of the arm at a lateral pad-abutting point not covered by the brace, thereby providing at least three access/observation points adjacent to the pad at the anticubital fossa. And of these embodiments it is further preferred that the strapping includes a figure-8 securement strap having a first loop, a second loop and a pad-attachment portion, and still further preferred that, in the mounted position, the first loop is disposed to fit around an back-side part of the upper arm, the second loop is disposed to fit around a back-side part of the lower arm, and the elbow is not covered by the brace.

It is still further preferred that the pad includes a hole defined by a topside opening, an underside opening and a passage therebetween, and the figure-8 securement strap includes a pass-through portion that passes through the hole. In embodiments including such a hole, it is additionally preferred that the pad includes two holes. More specifically, it is preferred that the pad includes a first hole defined by a first topside opening, a first underside opening and a passage therebetween, and a second hole defined by a second topside opening, a second underside opening and a passage therebetween; in such embodiments the topside includes a middle area between the first and second topside openings, the figure-8 securement strap passes through the two holes and includes an intersection point over the middle area, with a first intersecting portion of the strap crossing over a second intersecting portion of the strap at the intersection point.

In certain preferred embodiments of this invention, the brace includes a strap-adjuster attached to one of two strap loops. In such embodiments, it is preferable for the strap-adjuster to have a fixed point of attachment on one of the first loop and second loop, and for the figure-8 securement strap to include at least 2 inches of strap length between the fixed point and the intersecting point. In certain preferred embodiments, the strap-adjuster is a ring of nylon material. Such ring may have any of a variety of shapes, including for example rectangular, circular or D-shaped, but in most highly preferred embodiments the ring is rectangular.

In some preferred embodiments, the brace includes a figure-8 securement strap with a self-adhesive surface, which facilitates user-selected strap-length adjustment. In highly preferred embodiments, such a strap includes includes both a hook-type fabric surface and a loop-type fabric surface, and the hook-type fabric surface is disposed to securely but removably mate with the loop-type fabric surface.

In preferred embodiments of this invention, including embodiments not adapted for use with the human arm, the substantially planar underside of the pad is of a resiliently deformable material. And in highly preferred embodiments having an underside of such material, the pad is a unitary object of a single material.

Among embodiments having a figure-8 securement strap, it is further preferred that the strap be of an elastic material.

The method of this invention is stated with respect to a human arm, and more particularly with respect to a catheter subcutaneously present in the antecubital fossa of the arm. The method includes providing a brace having a disk-like pad including a peripheral edge therearound, a topside and a substantially planar underside; and strapping configured to hold the pad over tissue surrounding the subcutaneous catheter while maintaining substantial planarity of the underside and a region of tissue thereunder, and securing such strapping to the arm with the pad at the antecubital fossa of the arm.

In certain preferred embodiments of this inventive method, the strapping used is a figure-8 securement strap with first and second loops and a point of attachment to the pad, and the method includes disposing one of the loops around a back-side part of the upper arm and disposing the other loop around a back-side part of the lower arm, so that the brace is mounted on the arm in such a configuration that the elbow is uncovered. Highly preferred embodiments of the invention include providing at least three access/observation points adjacent to the pad at the anticubital fossa by the configuration of the brace and the disposing of the strapping on the arm.

As used herein with regard to the pad, the term “disk-like” describes a three-dimensional shape having a maximum thickness dimension less than the two other maximum dimensions of the shape perpendicular thereto, an underside of the pad being sized to fit on one side of a human arm at the elbow pit.

As used herein with regard to the underside of the pad, the term “substantially planar” means sufficiently in a single plane that, upon arm bending, any deformation of tissue upon arm bending is primarily caused by tissue interaction with opposite edge-adjacent portions of the underside, leaving tissue under a middle portion of the underside relatively undeformed along the length of the arm. In preferred embodiments, the pad material is resiliently deformable, a term which in this context is consistent with the substantial-planarity-related function described in this paragraph.

As used herein with regard to the underside, the term “middle portion” refers to a region of the underside having a cross-dimension along the length of the arm which is a majority of the cross-dimension along the entire underside in that direction.

### BRIEF DESCRIPTION OF THE DRAWINGS

FIG. 1 is a perspective view of a brace in accordance with this invention, in an unmounted position.

FIG. 2 is a fragmentary underside view of the brace of FIG. 1.

FIG. 3 is an underside view of the pad of the brace of FIG. 1, without the strapping.

FIG. 4 is a perspective view of the pad of the brace of FIG. 1, without the strapping, drawn in a manner showing primarily the topside of the pad.

FIG. 5 is a view of the brace of FIG. 1 in a mounted position on a human arm, with the pad at the antecubital fossa.

### DETAILED DESCRIPTION OF PREFERRED EMBODIMENTS

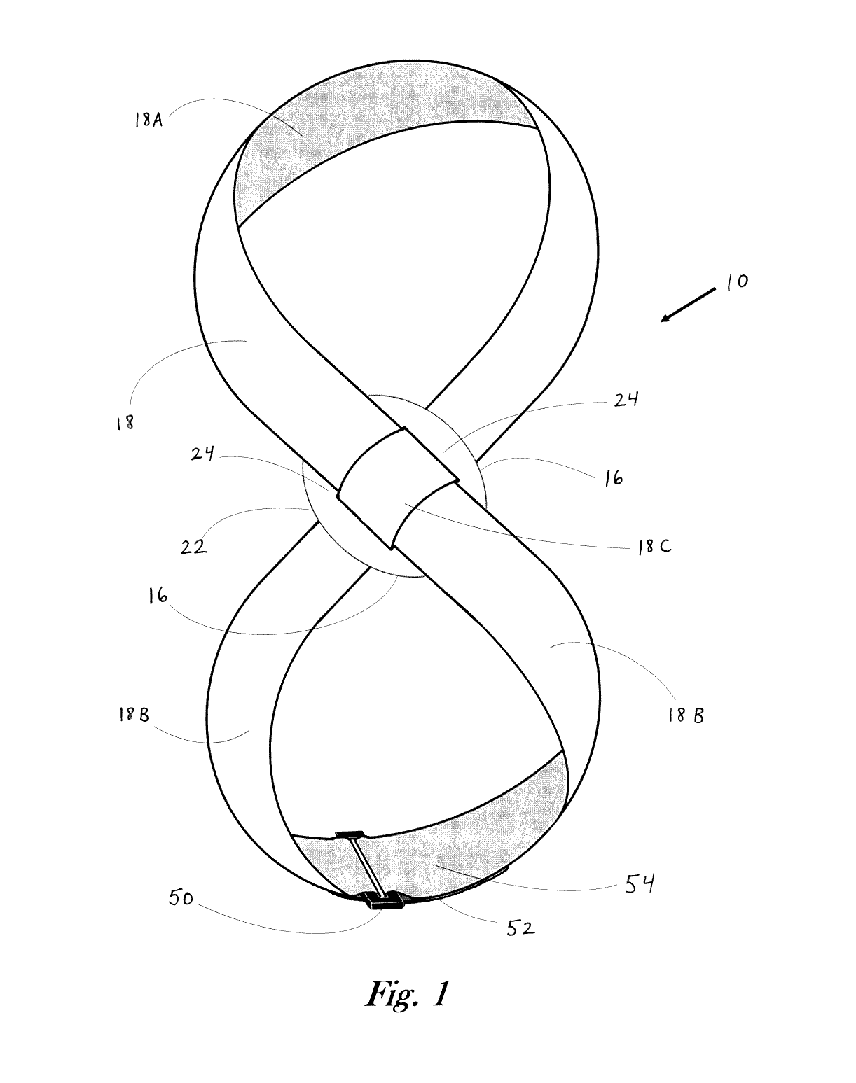

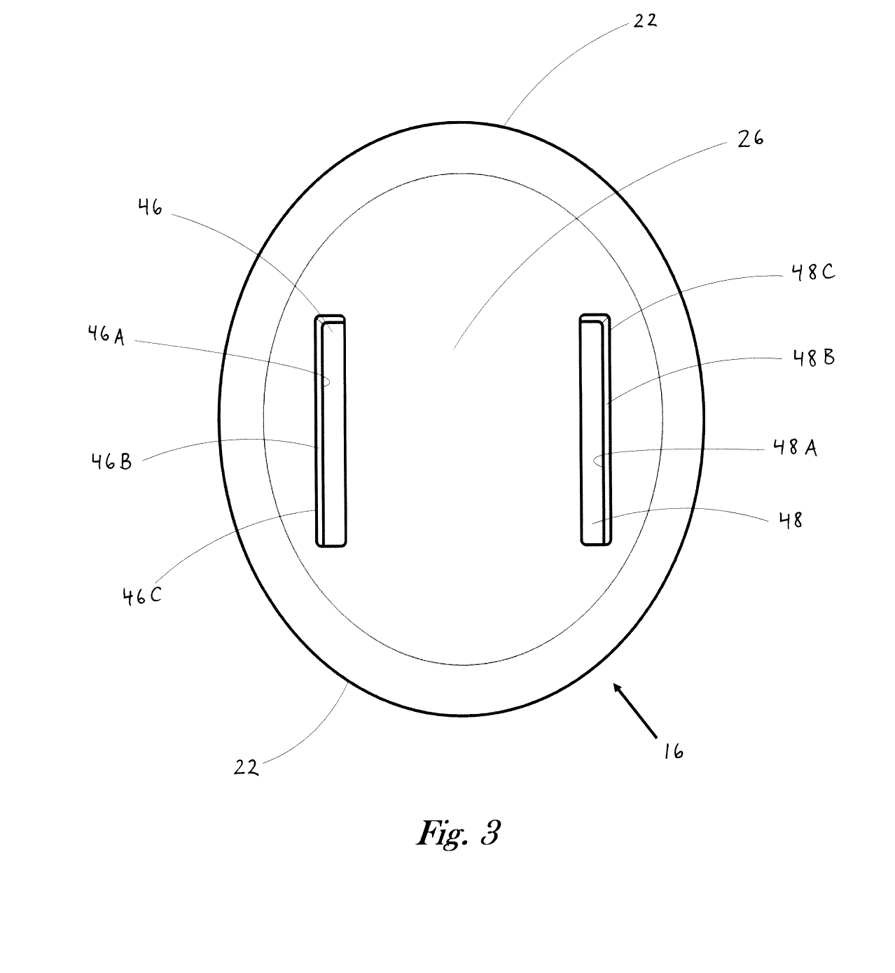

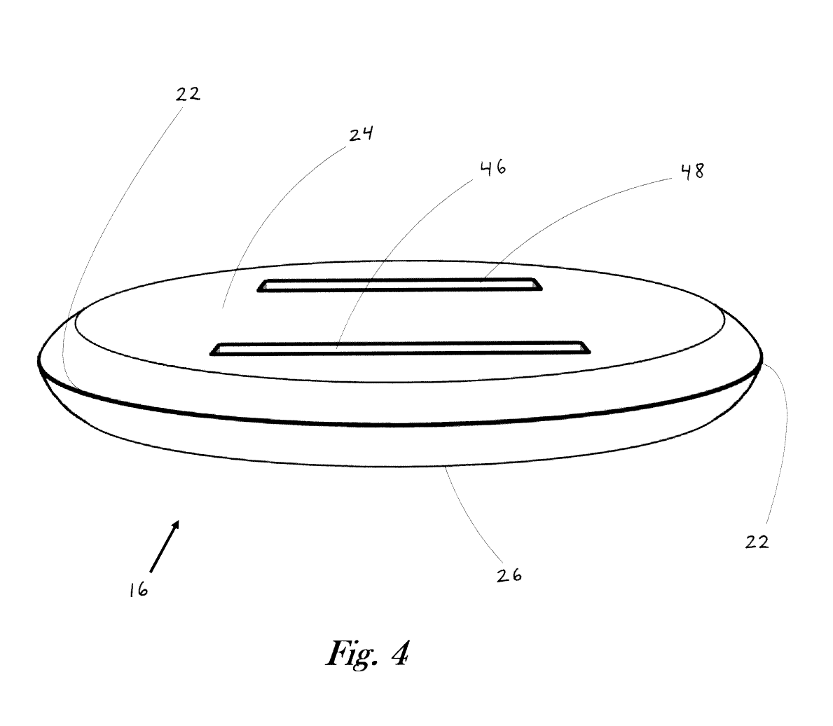

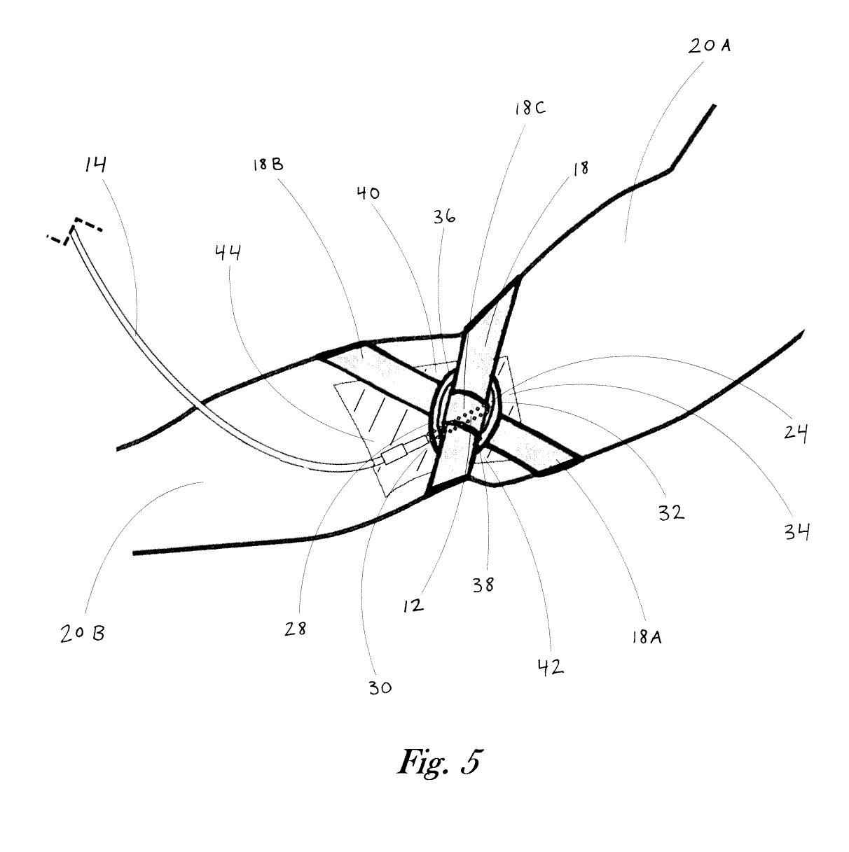

FIGS. 1-5 illustrate a preferred anti-bend catheter brace 10 in accordance with this invention. Catheter brace 10 inhibits subcutaneous bending of a catheter 12, represented in FIG. 5 by dotted lines that indicate the subcutaneous presence of catheter 12. Catheter 12 is connected to tube 14 through which fluids flow to catheter 12.

Brace 10 includes a disk-like pad 16 and strapping 18 for connection to a patient's upper arm 20A and lower 20B, as seen in FIG. 5. Pad 16 has a peripheral edge 22 therearound, a topside 24 and a substantially planar underside 26. Strapping 18 is configured to hold the pad over tissue surrounding subcutaneous catheter 12 while maintaining substantial planarity of underside 26 and a region of tissue thereunder.

Strapping 18 is in a figure-8 form which includes a first loop 18A, a second loop 18B, and a pad-attachment portion. First loop 18A is disposed to fit around an back-side part of upper arm 20A, second loop 18B is disposed to fit around a back-side part of lower arm 20B, and the elbow is not covered by any part of brace 10, including the strapping. Such figure-8 configuration is particularly useful in providing ready access of medical personnel to the antecubital fossa for purposes of observation and other care.

Before describing holes in pad 16 that accommodate the figure-8 strapping, FIG. 5 will be referred to for purposes of describing portions of pad 16 pertaining to ready access. Pad 16 has a lower-arm-adjacent portion 28 abutting an adjacent region 30 of lower arm 20B at a lower-arm pad-abutting point not covered by brace 10. Likewise, pad 16 has an upper-arm-adjacent portion 32 abutting an adjacent region 34 of the upper arm 20A at an upper-arm pad-abutting point not covered by brace 10. And pad 16 also has lateral portions 36 and 38 which abut adjacent arm regions 40 and 42, respectively, arm regions not covered by brace 10. Thus, using brace 10 not only serves to inhibit subcutaneous bending of catheter 12, but provides four access/observation points adjacent to pad 16 at the anticubital fossa. In FIG. 5, the antecubital fossa of the arm, including arm regions 30, 34, 40 and 42, are covered by clean-field plastic dressing 44, but are not covered by any part of brace 10.

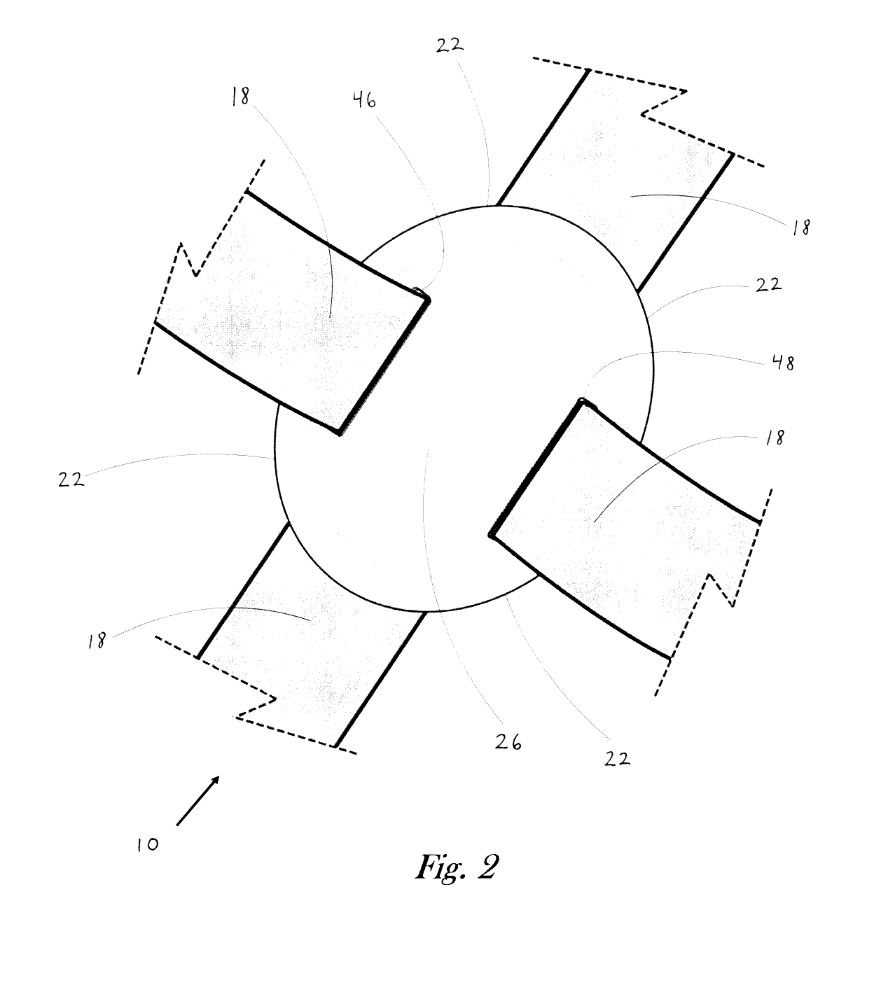

Holes in pad 16 are seen in each of FIGS. 1-5, but FIGS. 2-4 are particularly useful in describing such holes. Formed in pad 16 is a first hole 46 defined by a first topside opening 46A, a first underside opening 46C, and a passage 46B therebetween. Also formed in pad 16 is a second hole 48 defined by a second topside opening 48A, a second underside opening 48C, and a passage 48B therebetween. As described best by reference to FIGS. 1 and 2, the figure-8 strap of strapping 18 extends from first loop 18A through first hole 46 from underside 26 to topside 24, then passing over another portion the strap and then through second hole 48 from topside 24 to underside 26. From there the strap continues to form second loop 18B which is configured to fit around lower arm 20B, and then extends across topside 24 of pad 16 including across the region of topside 24 between holes 46 and 48 and at such point being under another portion of the strap. Such intersecting and hole-engaging areas of the strap form pad attachment portion 18C of strapping 18. From that point, the strap extends to form upper loop 18A of strapping 18, which is configured to fit around upper arm 20A. As can readily be seen from the figures, strapping 18 forms a continuous FIG. 8 pattern that includes upper and lower loops 18A and 18B.

Brace 10 also includes a strap-adjuster 50 attached to second loop 18B, allowing adjustment of strap length, conveniently at the lower arm of the patient. Strap adjuster 50 is a ring of nylon material, and is in a rectangular shape. A strap-to-strap self-adhesive portion of strapping 18 is located at regions 52 and 54 of strapping 18. The self-adhesive portion includes a loop-type fabric surface and the hook-type fabric surface (such as Velcro®), which serves to securely but removably mate the strap portions to one another.

Strapping 18 is of any of a wide variety of suitable elastic strap materials. Pad 16 is a unitary object of a medically-acceptable resiliently deformable material. The term “resiliently deformable” has a meaning described near the end of the “Summary of the Invention” portion of this document.

Brace 10 as described herein is suitable in connection with the method described above.

While the principles of this invention have been described in connection with specific embodiments, it should be understood clearly that these descriptions are made only by way of example and are not intended to limit the scope of the invention. Numerous alternative configurations providing all the benefits of the invention are possible.

1. A brace for inhibiting subcutaneous bending of an inserted catheter, the brace comprising:
a disk-like pad including a peripheral edge therearound, a topside and a substantially planar underside; and
strapping configured to hold the pad over tissue surrounding the subcutaneous catheter while maintaining substantial planarity of the underside and a region of tissue thereunder.

2. The brace of claim 1 adapted for use with a human arm, wherein the brace is movable between an unmounted position in which the brace is not attached to the arm and a mounted position in which the brace is secured to the arm with the pad at the antecubital fossa of the arm.

3. The brace of claim 2 wherein, in the mounted position, the pad has:
a lower-arm-adjacent portion abutting an adjacent region of the lower arm at a lower-arm pad-abutting point not covered by the brace;
an upper-arm-adjacent portion abutting an adjacent region of the upper arm at an upper-arm pad-abutting point not covered by the brace; and
at least one lateral portion abutting adjacent region of the arm at a lateral pad-abutting point not covered by the brace,
thereby providing at least three access/observation points adjacent to the pad at the anticubital fossa.

4. The brace of claim 3 wherein the strapping including a figure-8 securement strap having a first loop, a second loop and a pad-attachment portion.

5. The brace of claim 4 wherein, in the mounted position, the first loop is disposed to fit around a back-side part of the upper arm and the second loop is disposed to fit around a back-side part of the lower arm, the elbow not being covered by the brace.

6. The brace of claim 5 wherein the pad includes a hole defined by a topside opening, an underside opening and a passage therebetween, the pad-attachment portion of the figure-8 securement strap including a pass-through portion that passes through the hole.

7. The brace of claim 6 wherein the pad includes (a) a first hole defined by a first topside opening, a first underside opening and a passage therebetween, and (b) a second hole defined by a second topside opening, a second underside opening and a passage therebetween, the topside including a middle area between the first and second topside openings, the figure-8 securement strap passing through the two holes and including an intersection point over the middle area, a first intersecting portion of the strap crossing over a second intersecting portion of the strap at the intersection point.

8. The brace of claim 5 wherein the brace includes a strap-adjuster attached to one of the first loop or second loop.

9. The brace of claim 8 wherein the strap-adjuster has a fixed point of attachment on one of the first loop and second loop, and the figure-8 securement strap includes at least 2 inches of strap length between the fixed point and the intersecting point.

10. The brace of claim 8 wherein the strap-adjuster is a ring of nylon material.

11. The brace of claim 5 wherein the figure-8 securement strap includes at least one self-adhesive surface, the self-adhesive surface facilitating user-selected strap-length adjustment.

12. The brace of claim 5 wherein the figure-8 securement strap includes a hook-type fabric surface and a loop-type fabric surface, the hook-type fabric surface being disposed to securely but removably mate with the loop-type fabric surface.

13. The brace of claim 1 wherein the substantially planar underside is of a resiliently deformable material.

14. The brace of claim 13 wherein the pad is a unitary object of a single material.

15. The brace of claim 4 wherein the figure-8 securement strap is of an elastic material.

16. A method of inhibiting subcutaneous bending of a catheter subcutaneously present in the antecubital fossa of a human arm, the method comprising: providing a brace having (a) a disk-like pad including a peripheral edge therearound, a topside and a substantially planar underside, and (b) strapping configured to hold the pad over tissue surrounding the subcutaneous catheter while maintaining substantial planarity of the underside and a region of tissue thereunder, and securing the strapping to the arm with the pad at the antecubital fossa of the arm.

17. The method of claim 16 wherein the strapping is a figure-8 securement strap having first and second loops and a pad-attachment portion, and the method includes disposing one of the loops around a back-side part of the upper arm and disposing the other loop around a back-side part of the lower arm, the elbow not being covered by the brace.

18. The method of claim 17 including providing at least three access/observation points adjacent to the pad at the anticubital fossa by the configuration of the brace and the disposing of the strapping on the arm.
